(19) United States
(12) Patent Application Publication
(10) Pub. No.: US 2022/0139563 A1
(43) Pub. Date: May 5, 2022

(54) APPARATUS AND METHOD FOR MANAGING CIRCADIAN RHYTHM BASED ON FEEDBACK FUNCTION

(71) Applicants: HUCIRCADIAN CO., LTD., Seoul (KR); KOREA UNIVERSITY RESEARCH AND BUSINESS FOUNDATION, Seoul (KR)

(72) Inventor: Heon Jeong LEE, Seoul (KR)

(21) Appl. No.: 17/474,616

(22) Filed: Sep. 14, 2021

(30) Foreign Application Priority Data
Nov. 5, 2020 (KR) 10-2020-0146801

(51) Int. Cl. G16H 50/30; G16H 40/63

(52) U.S. Cl. CPC G16H 50/30; G16H 40/63

(57) ABSTRACT

An apparatus and method for managing a circadian rhythm based on a feedback function are disclosed. The circadian rhythm management method based on the feedback function according to the exemplary embodiment includes collecting feature information including a heart rate information, light exposure information, sleep information, and step information for a user, calculating a health score for the user based on the feature information, and providing feedback information including a behavior pattern to improve the health score based on the calculated health score through a user terminal of the user.

### BACKGROUND

### Field

The present disclosure relates to an apparatus and a method for managing a circadian rhythm based on a feedback function.

### Description of the Related Art

Excessive stress and mental health problems of modern people are significantly related to each other, but the stress and the mental health problems are relayed by the disturbance of life rhythm.

Outbreak or recurrence of various mental disorders such as insomnia, depression, or bipolar disorder which are commonly experienced by the stress is closely related to the disturbance of the circadian rhythm. Therefore, importance of chronotherapy which controls a circadian rhythm is being emphasized.

However, due to the lack of techniques for controlling circadian rhythms and detecting a change thereof, it is difficult to effectively perform a preventive treatment of the insomnia and effectively prevent recurrent depression or bipolar disorder so that it is difficult to prevent and treat the disturbance of mental health.

In the meantime, in accordance with the rapid growth of the digital health care technology and markets, applications related to the mental health care, such as mood, cognition, or sleep are being developed. Further, in recent years, various user log data which is called digital phenotype may be collected by smart phones, wearable devices, or the like. Therefore, it is expected that the log data is used to not only simply acquire user's biometric data, but also analyze and manage the circadian rhythm related to the user's mental health.

A related art of the present disclosure is disclosed in Korean Registered Patent Publication No. 10-1712281.

### SUMMARY

An object to be achieved by the present disclosure is to solve the above-described problems of the related art and provide an apparatus and a method for managing a circadian rhythm based on a feedback function which collect various log data (feature information) associated with a user's life pattern to evaluate health scores of the user from various aspects and provide an appropriate feedback including a behavior pattern to improve the health score based on the evaluated health score.

However, objects to be achieved by various embodiments of the present disclosure are not limited to the technical objects as described above and other technical objects may be present.

As a technical means to achieve the above-described technical object, according to an aspect of the present disclosure, a circadian rhythm management method based on a feedback function includes collecting feature information including a heart rate information, light exposure information, sleep information, and step information for a user; calculating a health score for the user based on the feature information; and providing feedback information including a behavior pattern to improve the health score based on the calculated health score through a user terminal of the user.

Further, in the collecting of feature information, the light exposure information and the step information are separately collected for each of a plurality of time slots which is divided in advance.

Further, the circadian rhythm management method based on a feedback function according to the exemplary embodiment of the present disclosure may include generating mood change prediction information of the user corresponding to the feature information based on a prediction model which is trained in advance to output mood change prediction information corresponding to the input feature information and providing the mood change prediction information through the user terminal.

Further, in the providing of mood change prediction information, a virtual avatar whose outer appearance is determined in response to the mood change prediction information is displayed on the user terminal.

Further, the heart rate information may include an acrophase, an amplitude, a mesor of the vibration, and a coefficient of determination in a pulse fitting curve derived based on the pulse rate per unit time of the user.

Further, the light exposure information may include a light exposure amount during the daytime and a light exposure amount during the bedtime.

Further, the sleep information may include a sleep length, a sleep quality, a sleep onset deviation, and a sleep offset deviation.

Further, the step information may include cumulative step counts during the daytime and cumulative step counts during the bedtime.

Further, the calculating of the health score may include calculating a heart rate related score of the user based on at least one of the acrophase, the amplitude, the mesor of the vibration, and the coefficient of determination, calculating a light exposure related score based on at least one of the light exposure amount during the daytime and the light exposure amount during the bedtime, calculating a sleep related score based on at least one of the sleep length, the sleep quality, the sleep onset deviation, and the sleep offset deviation, and calculating an activity related score based on at least one of the cumulative step counts during the daytime and the cumulative step counts during the bedtime.

Further, the circadian rhythm management method based on a feedback function according to the exemplary embodiment of the present disclosure may include providing a predetermined warning alarm to the user terminal when the health score is deteriorated to be lower than a predetermined level.

Further, in the providing of a predetermined warning alarm, when an average value of the heart rate related score, the light exposure related score, the sleep related score, and the activity related score is lower than a predetermined first threshold value, the warning alarm may be provided.

Further, in the providing of the warning alarm, when the average value for a predetermined monitoring period is maintained to be lower than a predetermined second threshold value, the warning alarm is provided.

Further, the second threshold value may be set to be higher than the first threshold value.

Further, in the collecting of feature information, the feature information may be collected from at least one of the user terminal and a wearable device which is worn on a body of the user.

Further, the circadian rhythm management method based on a feedback function according to the exemplary embodiment of the present disclosure may include collecting wearing hour information of the wearable device and providing feedback information based on the wearing hour information to the user terminal.

In the meantime, according to another aspect of the present disclosure, a circadian rhythm management apparatus based on a feedback function may include a collecting unit which collects feature information including a heart rate information, light exposure information, sleep information, and step information for a user, a score calculating unit which calculates a health score for the user based on the feature information, and a feedback providing unit which provides feedback information including a behavior pattern to improve the health score based on the calculated health score through a user terminal of the user.

Further, the circadian rhythm management apparatus based on a feedback function according to the exemplary embodiment of the present disclosure may include a mood predicting unit which generates a mood change prediction information corresponding to the feature information based on a prediction model trained in advance to output mood change prediction information corresponding to the input feature information and provides the mood change prediction information by means of the user terminal.

Further, the score calculating unit calculates a heart rate related score of the user based on at least one of the acrophase, the amplitude, the mesor of the vibration, and the coefficient of determination, calculates a light exposure related score based on at least one of the light exposure amount during the daytime and the light exposure amount during the bedtime, calculates a sleep related score based on at least one of the sleep length, the sleep quality, the sleep onset deviation, and the sleep offset deviation, and may calculate an activity related score based on at least one of the cumulative step counts during the daytime and the cumulative step counts during the bedtime.

Further, the circadian rhythm management apparatus based on a feedback function according to the exemplary embodiment of the present disclosure may include an alarm unit which provides a predetermined warning alarm to the user terminal when the health score is deteriorated to be lower than a predetermined level.

The above-described solving means are merely illustrative but should not be construed as limiting the present disclosure. In addition to the above-described embodiments, additional embodiments may be further provided in the drawings and the detailed description of the present disclosure.

According to the present disclosure, it is possible to provide an apparatus and a method for managing a circadian rhythm based on a feedback function which collect various log data (feature information) associated with a user's life pattern to evaluate health scores of the user from various aspects and provide an appropriate feedback including a behavior pattern to improve the health score based on the evaluated health score.

However, the effect which may be achieved by the present disclosure is not limited to the above-described effects, there may be yet another effects.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a schematic diagram of a digital health care system including a circadian rhythm management apparatus based on a feedback function according to an exemplary embodiment of the present disclosure;

FIG. 2 is a table illustrating feature information collected for a user;

FIG. 3 is a view illustrating a pulse fitting curve related to pulse information of a user;

FIG. 4 is a view illustrating an interface expressed to provide feedback information through a user terminal;

FIG. 5 is a view illustrating an interface expressed to provide mood change prediction information of the user through a user terminal;

FIG. 6 is a view illustrating an interface expressed to provide evaluation information obtained by collecting user's health scores through a user terminal;

FIGS. 7A and 7B are views illustrating a result of comparing feature information and mood episodes between a user group to which a feedback based circadian rhythm management of the present disclosure is applied and a user group to which the circadian rhythm management is not applied, as an experimental embodiment related to a circadian rhythm management apparatus based on a feedback function according to an exemplary embodiment of the present disclosure;

FIG. 8 is a view illustrating a result of comparing a wearing rate of a wearable device between a user group to which a feedback based circadian rhythm management of the present disclosure is applied and a user group to which the circadian rhythm management is not applied, as an experimental embodiment related to a circadian rhythm management apparatus based on a feedback function according to an exemplary embodiment of the present disclosure;

FIG. 9 is a schematic diagram of a circadian rhythm management apparatus based on a feedback function according to an exemplary embodiment of the present disclosure; and

FIG. 10 is a flowchart of an operation of a circadian rhythm management method based on a feedback function according to an exemplary embodiment of the present disclosure.

### DETAILED DESCRIPTION OF THE EMBODIMENT

Hereinafter, the present disclosure will be described more fully hereinafter with reference to the accompanying drawings, in which exemplary embodiments of the present disclosure are shown. However, the present disclosure may be realized in various different forms, and is not limited to the embodiments described herein. Accordingly, in order to clearly explain the present disclosure in the drawings, portions not related to the description are omitted. Like reference numerals designate like elements throughout the specification.

Throughout this specification that follow, when it is described that an element is “coupled” to another element, the element may be “directly coupled” to the other element or “electrically coupled” or “indirectly coupled” to the other element through a third element.

Through the specification of the present disclosure, when one member is located “on”, “above”, “on an upper portion”, “below”, “under”, and “on a lower portion” of the other member, the member may be adjacent to the other member or a third member may be disposed between the above two members.

In the specification of the present disclosure, unless explicitly described to the contrary, the word “comprise” and variations such as “comprises” or “comprising”, will be understood to imply the inclusion of stated elements but not the exclusion of any other elements.

The present disclosure relates to a circadian rhythm management apparatus and method based on a feedback function.

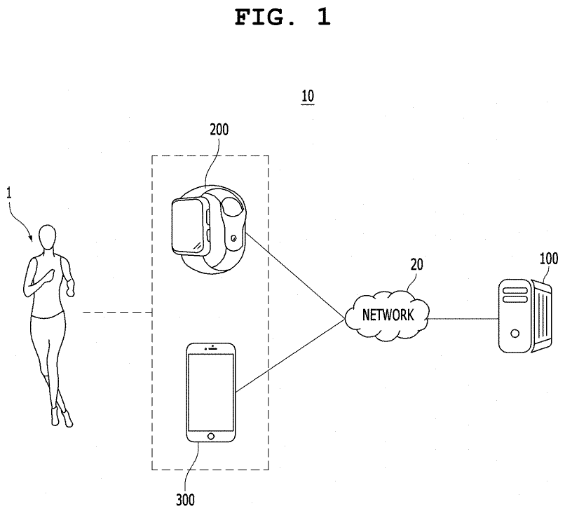

FIG. 1 is a schematic diagram of a digital health care system including a circadian rhythm management apparatus based on a feedback function according to an exemplary embodiment of the present disclosure.

Referring to FIG. 1, a digital health care system 10 according to an exemplary embodiment of the present disclosure may include a circadian rhythm management apparatus 100 based on a feedback function (hereinafter, simply referred to as a “circadian rhythm management apparatus 100”), a wearable device 200, and a user terminal 300.

The circadian rhythm management apparatus 100, the wearable device 200, and the user terminal 300 may communicate with each other by means of a network 20. The network 20 means a connection structure which allows information exchange between nodes such as terminals or servers. Examples of the network 200 include 3^{rd} generation partnership project (3GPP) network, a long term evolution (LTE) network, a 5G network, a world interoperability for microwave access (WIMAX) network, Internet, a local area network (LAN), a Wireless Local Area Network (LAN), a Wide Area Network (WAN), a personal area network (PAN), a Wi-Fi network, a Bluetooth network, a satellite broadcasting network, an analog broadcasting network, a digital multimedia broadcasting (DMB) network, and the like, but are not limited thereto. Further, the circadian rhythm management apparatus 100 according to the exemplary embodiment of the present disclosure may be implemented to be loaded (installed) in at least one of the wearable device 200 and the user terminal 300.

For example, the wearable device 200 may be a smart watch or a smart band which is worn on the wrist of a user to collect biometric information of the user, but is not limited thereto. As another example, the wearable device 200 may broadly refer to a device which is worn on various positions to acquire feature information of the user 1, such as a head-worn device, a strap type device, a garment-type device, or shoe-worn/foot pods device. Further, for example, the wearable device 200 may be Fitbit, Jawbone Up, Nike+ FuelBand, Apple Watch, Samsung Gear, or the like. Further, the wearable device 200 and the user terminal 300 may be interlinked based on the same account information for one user 1. Further, according to an implementation embodiment of the present disclosure, it may be understood that the wearable device 200 is included in the user terminal 300 in a broad sense.

For example, the user terminal 300 may include all kinds of wireless communication devices such as a smart phone, a smart pad, a tablet PC, a personal communication system (PCS), a global system for mobile communication (GSM), a personal digital cellular (PDC), a personal handyphone system (PHS), a personal digital assistant (PDA), an international mobile telecommunication (IMT)-2000, code division multiple access (CDMA)-2000, W-code division multiple access (W-CDMA), and a wireless broadband internet (Wibro) terminal.

The circadian rhythm management apparatus 100 may collect the feature information of the user 1 based on at least one of the user terminal 300 and the wearable device 200. Further, according to the exemplary embodiment of the present disclosure, the feature information collected by the circadian rhythm management apparatus 100 may include heart rate information, light exposure information, sleep information, and step information of the user 1.

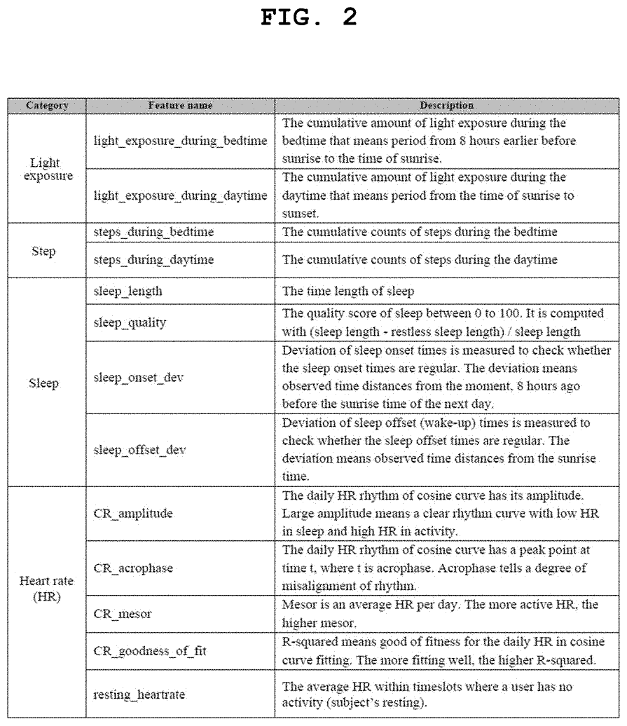

FIG. 2 is a table illustrating feature information collected for a user.

Referring to FIG. 2, the feature information collected for the user 1 may be classified into categories including light exposure information (light exposure), step information (step), sleep information (sleep), and heart rate information (HR) to be collected.

Further, according to the exemplary embodiment of the present disclosure, the circadian rhythm management apparatus 100 may collect the light exposure information and the step information separately for each of a plurality of time slots which is divided in advance. To be more specific, the plurality of time slots may be obtained by dividing one day into two time slots as a daytime and a bedtime or dividing one day into three time slots by eight hours based on a sunrise time or a sunset time, but it is not limited thereto. Further, when one day is divided into two time slots, the plurality of time slots is equally divided by 12 hours to be the daytime and the bedtime, but it is not limited thereto. Therefore, the plurality of time slots may be unequally divided in accordance with a region, the season, the date, and the climate (for example, the bedtime is from eight hours before the sunrise time to the sunrise time and the daytime is the remaining time slot (16 hours).

With regard to this, referring to FIG. 2, the circadian rhythm management apparatus 100 according to the exemplary embodiment of the present disclosure may collect the light exposure information and the step information based on two divided time slots (in other words, time slots divided into the daytime and the bedtime).

The feature information collected for every category described above will be described in more detail. The light exposure information category may include a light exposure amount during the daytime (light exposure during daytime) and a light exposure amount during the bedtime (light exposure during bedtime). Specifically, the light exposure amount during the bedtime is a light exposure amount collected during a time when the user 1 normally sleeps, and for example, light exposure information collected for a time slot from eight hours before the sunrise time to the sunrise time in one day. Further, the light exposure amount during the daytime may be light exposure information collected for a remaining time slot other than the time slot from eight hours before the sunrise time to the sunrise time. Specifically, with regard to the light exposure information, light is the most important trigger which synchronizes a circadian rhythm of all the living things on earth as well as the humans. Therefore, the health score (specifically, light exposure related score) to be described below may be evaluated to be bad as the light exposure amount during the bedtime is increased and the health score may be evaluated to be good as the light exposure amount during the daytime is increased.

Next, the step information category may include cumulative steps during the daytime (steps during daytime) and cumulative steps during the bedtime (steps during bedtime). Here, the daytime and the bedtime may be divided by the same manner as determined in the light exposure information category. Specifically, the cumulative step counts during the daytime are information obtained by accumulating and counting the step counts of the user 1 collected for the daytime and the cumulative step counts during the bedtime are information obtained by accumulating and counting the step counts of the user 1 collected for the bedtime. The health score (specifically, activity related score) to be described below may be evaluated to be good when the user 1 maintains a healthy circadian rhythm and shows a life pattern which clearly distinguishes the sleep state from the wake state. For example, the larger the cumulative step counts during the daytime, the better the health score to be evaluated and the larger the cumulative step counts during the bedtime, the worse the health score to be evaluated.

Next, the sleep information (Sleep) category may include a sleep length (sleep length), a sleep quality (sleep quality), a sleep onset deviation (sleep onset dev), and a sleep offset deviation (sleep offset dev) information. Specifically, the sleep length may refer to a time from a timing that the sleep starts to a timing that the sleep ends on the corresponding day. According to the exemplary embodiment of the present disclosure, the timing that the sleep of the user 1 starts or the timing that the sleep of the user 1 ends may be determined by a user input (for example, a user input indicating to start the sleep before the sleep is applied or a user input indicating to end the sleep and wake up after waking-up is applied) which is directly applied to at least one of the user terminal 300 and the wearable device 200 by the user 1. Alternatively, the timing that the sleep of the user 1 starts or the timing that the sleep of the user 1 ends may be deduced by considering the remaining feature information such as the step information or the light exposure information of the user 1 acquired from at least one of the user terminal 300 and the wearable device 200.

Further, according to the exemplary embodiment of the present disclosure, the sleep quality may be calculated by the following Equation 1.

(sleep length−restless sleep length)/sleep length [Equation 1]

Here, the “sleep length” which is the entire sleeping hours is feature information corresponding to the above-described sleep length and “restless sleep length” may refer to a time which is evaluated that the user 1 has a restless sleep state. For example, the sleep quality may be information collected when the user 1 takes the sleep while wearing a wearable device 200 from the wearable device 200 having a function which is capable of evaluating the sleep state of the user 1 or how much the user 1 turns over during the sleep.

Further, the sleep onset deviation information (sleep_onset_dev) is feature information indicating whether the user 1 regularly starts the sleep and may be a deviation between a reference timing (for example, eight hours before the sunrise time to the sunrise time) and the sleep onset time of the user 1. Further, the sleep offset deviation information (sleep_offset_dev) is feature information indicating whether the user 1 regularly ends the sleep and may be a deviation between a reference timing (for example, the sunrise time) and the sleep offset time of the user 1.

Specifically, with regard to the sleep information category, as the user sufficiently and regularly sleeps, the health score (specifically, a sleep related score) may be calculated to be good.

Next, the heart rate HR information category may include an acrophase (CR_acrophase), an amplitude (CR_amplitude), a mesor of the vibration (CR_mesor), a coefficient of determination (CR_goodness_of_fit), and a resting heartrate (resting_heartrate) in a pulse fitting curve derived based on a pulse rate per unit time of the user 1.

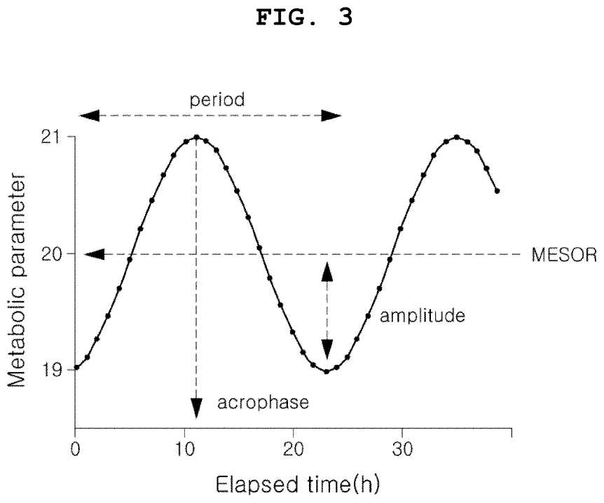

FIG. 3 is a view illustrating a pulse fitting curve related to heart rate information of a user.

Referring to FIG. 3, the heart rate HR of the user includes main rhythm information and the heart rate of the user tends to be lowered during the sleep and increase during the activity. Therefore, the pulse fitting curve of the user who sleeps during the bedtime and does many activities during the daytime shows an ideal cosine curve similar to S.

Specifically, the amplitude (CR amplitude) in the pulse fitting curve refers to an amplitude in a pulse fitting curve having a cosine curve shape. The larger the amplitude, the clearer the heart rate rhythm. With regard to this, when the amplitude is relatively large, the user 1 may be in activity and when the amplitude is relatively small, the user 1 may be in sleep.

Further, the acrophase (CR acrophase) refers to how much the heart rate rhythm which quantifies the phenomenon that the circadian rhythm (for example, the pulse fitting curve, etc.) is pushed or pulled on a time axis is misaligned. With regard to this, it is proven that a phenomenon that the circadian rhythm is pushed or pulled and the onset of a mood disorder such as manic or depression are correlated.

Further, the coefficient of the determination (CR goodness of fit) indicates how well the pulse fitting curve faithfully represents original sample data and may have a value between 0 and 1. As the coefficient of the determination is closer to 1, the cosine curve is more surely fitted. In contrast, as the coefficient is closer to 0, it means that the cosine curve is not satisfactorily fitted. That is, as the pulse fitting curve is well fitted to the shape of the cosine curve, a value of R-square R² may be increased.

Further, the resting heartrate (resting heartrate) may refer to an average heart rate in a time slot when there is no activity of the user 1. Specifically, the resting heartrate may increase when the user 1 is stressed or in an anxious state, so that the resting heartrate may be utilized as the feature information related to the mood state of the user 1.

Further, as described above, the circadian rhythm management apparatus 100 may calculate a health score H-score of the user 1 based on the feature information of the user 1 collected from various aspects. Specifically, the circadian rhythm management apparatus 100 may include a heart rate related score (CR_h_score), a light exposure related score (LE_h_score), a sleep related score (SL_h_score), and an activity related index (ACT_h_score).

Further, according to the exemplary embodiment of the present disclosure, the circadian rhythm management apparatus 100 may calculate the heart rate related score (CR_h_score) of the user 1 based on at least one of the acrophase, the amplitude, the mesor of the vibration, and the coefficient of determination. To be more specific, the circadian rhythm management apparatus 100 according to the exemplary embodiment of the present disclosure may compute the heart rate related score (CR_h_score) based on the amplitude and the acrophase included in the heart rate information by the following Equation 2.

CR_h_score=0.5*N(CR_amplitude)+0.5*(100−N(CR_acrophase) [Equation 2]

Here, CR amplitude is an amplitude included in the heart rate information and the CR acrophase may be an acrophase included in the heart rate information.

Further, N(x) may be a function of normalizing the input (the amplitude and the acrophase in Equation 2) to any one of values between 0 and 100. For example, within an observable range, N (x) for a minimum value is 0, N (x) for an average is 50, and N(x) for a maximum value may be 100.

For example, according to the exemplary embodiment of the present disclosure, the circadian rhythm management apparatus 100 may calculate a light exposure related score (LE_h_score) based on at least one of a light exposure amount during the daytime and a light exposure amount during the bedtime. Specifically, the circadian rhythm management apparatus 100 may calculate a light exposure related score (LE_h_score) by the following Equation 3.

LE_h_score=0.5*(100−N(light_exposure_during_bedtime)+0.5*N(light_exposure_during_daytime) [Equation 3]

Here, light exposure during bedtime is a light exposure amount during the bedtime included in the light exposure information, light exposure during daytime is a light exposure amount during the daytime included in the light exposure information, and N(x) may be the above-described normalization function.

Further, according to the exemplary embodiment of the present disclosure, the circadian rhythm management apparatus 100 may calculate a sleep related score (SL_h_score) based on at least one of a sleep length, a quality of sleep, a sleep onset deviation, and a sleep offset deviation. Specifically, the circadian rhythm management apparatus 100 according to the exemplary embodiment of the present disclosure may calculate the sleep related score (SL_h_score) based on the quality of sleep, the sleep onset deviation, and the sleep offset deviation by the following Equation 4.

SL_h_score=0.5*N(sleeep_efficiency)+0.25*(100−N(sleep_onset_dev))+0.25*(100−N(sleep_offset_dev)) [Equation 4]

Here, sleep efficiency is a quality of sleep included in the sleep information, sleep onset dev is a sleep onset deviation included in the sleep information, sleep offset dev is a sleep offset deviation included in the sleep information, and N(x) may be the above-described normalization function.

Further, according to the exemplary embodiment of the present disclosure, the circadian rhythm management apparatus 100 may calculate an activity related score (ACT_h_score) based on at least one of cumulative step counts during the daytime and cumulative step counts during the bedtime.

Specifically, the circadian rhythm management apparatus 100 according to the exemplary embodiment of the present disclosure may calculate an activity related score (ACT_h_score) based on the cumulative step counts during the daytime and the cumulative step counts during the bedtime by the following Equation 5.

ACT_h_score=0.5*(100−N(steps_during_bedtime))+0.5*N(steps_during_daytime) [Equation 5]

Here, steps during bedtime is step counts during the bedtime, steps during daytime is step counts during the daytime, and N(x) may be the above-described normalization function.

Further, according to the exemplary embodiment of the present disclosure, in the above-described Equations 2 to 5 for computing the health score of each category, a constant (for example, 0.5, 0.25, or the like) which is multiplied with the feature information may be set to various values based on the personalized characteristic of the user 1 so that a user-customized health score may be computed. For example, the specific constant applied to the above-described Equations 2 to 5 may be set based on the gender, the age, a body type, BMI information, a disease history, and a mood disorder of the user.

Further, the circadian rhythm management apparatus 100 may provide feedback information including a behavioral pattern for improving a health score H-score based on the calculated health score H-score through at least one of the user terminal 300 and the wearable device 200 of the user 1. Here, the feedback information may refer to a coaching message including a behavior modification guide which is recommended to the user 1 to improve the health score when at least one of the health scores of the plurality of categories is evaluated to be equal to or lower than a predetermined level.

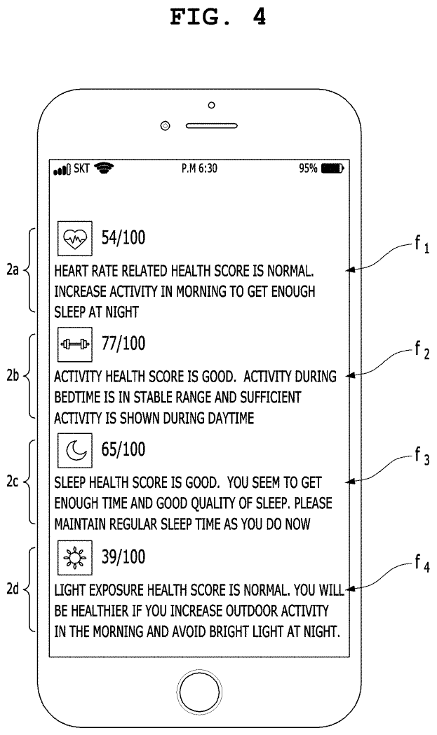

FIG. 4 is a view illustrating an interface expressed to provide feedback information through a user terminal.

Referring to FIG. 4, the circadian rhythm management apparatus 100 displays the calculated health score for every category (specifically, the heart rate related score, the light exposure related score, the sleep related score, and the activity related score) as scores (points) and divides the area in which the feedback information is expressed so as to correspond to each category (see 2a, 2b, 2c, and 2d of FIG. 4). In other words, in each area which is divided so as to correspond to the category of the health score, feedback information corresponding to the health score for every category may be individually expressed (f₁, f₂, f₃, and f₄ of FIG. 4).

To be more specific, for example, referring to FIG. 4, the heart rate related score of the user 1 which is 54 points out of 100 points as a perfect score, the activity related score which is 77 points out of 100 points as a perfect score, the sleep related score which is 65 points out of 100 points as a perfect score, and the light exposure related score which is 39 points out of 100 points as a perfect score are displayed on the user terminal 300.

According to the exemplary embodiment of the present disclosure, the feedback information expressed in response to the calculated health score may be determined so as to correspond to a numerical range to which the digitized (scored) health score for every category belongs.

For example, when the calculated heart rate related score belongs to a relatively low numerical range in a predetermined numerical range, it is determined to include a guidance text including a behavior pattern for improving the heart rate related score such as “Heart rhythm health score is not good. Increase activity in the morning and get enough sleep at night”. When the activity related score belongs to a relatively low numerical range in a predetermined numerical range, it is determined to include a guidance text including a behavior pattern for improving the activity related score such as “Activity health score is not good. Increase activity in the morning and refrain from taking a nap.” When the sleep related score belongs to a relatively low numerical range in a predetermined numerical range, it is determined to include a guidance text including a behavior pattern for improving the sleep related score such as “Sleep health score is not good. Stay awake in the morning and increase outdoor activity. Refrain from taking a nap.” When the light exposure related score belongs to a relatively low numerical range in a predetermined numerical range, it may be determined to include a guidance text including a behavior pattern for improving the light exposure related score such as “Light exposure related score is not good. Increase outdoor activity in the morning and avoid light at night. Specifically, looking at smartphone at night is harmful.” However, needless to say, the above-described guidance text included in the feedback information may be determined in various contents depending on the implementation embodiment of the present disclosure.

Further, according to the exemplary embodiment of the present disclosure, the numerical range of the health score to determine the contents of the feedback information may be divided into three ranges including low, normal, and high levels or if necessary, may be more specifically divided into four or more of a plurality of levels. The number of levels which is divided for every category of the health score may vary.

Further, according to the exemplary embodiment of the present disclosure, the circadian rhythm management apparatus 100 individually generates the feedback information for every category and may be implemented to determine the contents of the feedback information generated for the health score corresponding to a specific category in consideration of the health score or the feature information corresponding to at least one of the remaining categories other than the specific category. For example, even though the sleep related score belongs to a low numerical range, the contents of the feedback information for the sleep related score may be changed in response to the numerical range to which the activity related score, the light exposure related score, and the like belong.

Further, according to the exemplary embodiment of the present disclosure, even though not illustrated in the drawings, when the feedback information is provided to the wearable device 200 with a limited display area, the circadian rhythm management apparatus 100 may operate so as not to express the feedback information for the health scores for all categories, but to selectively display feedback information only for a health score type which is evaluated to be equal to or lower than a predetermined level.

As another example, the circadian rhythm management apparatus 100 may operate to express feedback information corresponding to a category selected by the user 1 based on the user input applied to at least one of the wearable device 200 and the user terminal 300.

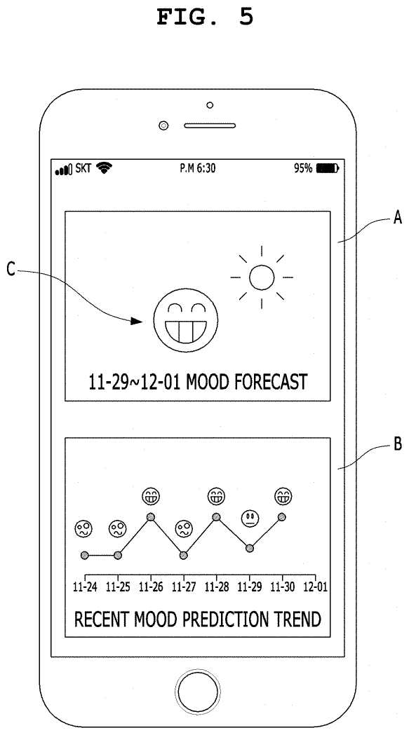

FIG. 5 is a view illustrating an interface expressed to provide mood change prediction information of the user through a user terminal.

Referring to FIG. 5, the circadian rhythm management apparatus 100 may generate mood change prediction information of the user 1 corresponding to feature information collected for the user 1, based on a prediction model which is trained in advance to output mood change prediction information corresponding to the input feature information.

Further, the circadian rhythm management apparatus 100 may provide the generated mood change prediction information to at least one of the user terminal 300 or the wearable device 200. For example, the circadian rhythm management apparatus 100 may display the mood change prediction information on an initial interface (for example, an interface displayed when an App which is associated with the circadian rhythm management apparatus 100 of the present disclosure is executed for the first time on the corresponding day) which is expressed when the application (App) or a program associated with the circadian rhythm management apparatus 100 disclosed in the present disclosure is executed, on at least one of the user terminal 300 and the wearable device 200.

Further, according to the exemplary embodiment of the present disclosure, the circadian rhythm management apparatus 100 may predict the mood change of the user 1 during a predetermined period based on feature information collected for the user 1, based on the prediction model implemented by machine learning based on learning data including the feature information collected for the plurality of users and the mood state information collected for a plurality of users. Here, the predetermined period may be three days from the present time (for example, referring to FIG. 5, a mood forecast between November 29 and December 1).

Further, with regard to the prediction model implemented by the machine learning, according to the exemplary embodiment of the present disclosure, the prediction model may be trained based on a supervised learning based random forest algorithm, but it is not limited thereto and in the present disclosure, various machine learning algorithm models which have been known in the related art or will be developed in the future may be applied.

Further, referring C in FIG. 5, the circadian rhythm management apparatus 100 may provide the mood change prediction information by displaying a virtual avatar whose outer appearance is determined in response to the mood change prediction information on at least one of the user terminal 300 and the wearable device 200. For helping the understanding, referring to C of FIG. 5, when the mood change prediction information predicted for the user 1 by the prediction model is predicted to be a good mood because it is predicted that the possibility of a mood episode is low, an avatar with a smiley face is displayed so that the user 1 may intuitively identify the present and future moods predicted in response to the feature information of the user. With regard to this, the circadian rhythm management apparatus 100 intuitively provides the mood change prediction information of the user 1 corresponding to the feature information collected for the user 1 to induce the user 1 to aware that the user's past behaviors (for example, a life pattern) may affect the user's future mood to make efforts to improve the behavior.

Further, referring to B (recent mood prediction trend) of FIG. 5, the circadian rhythm management apparatus 100 may provide the mood change prediction information of the user 1 predicted for a predetermined period as a time-sequential graph.

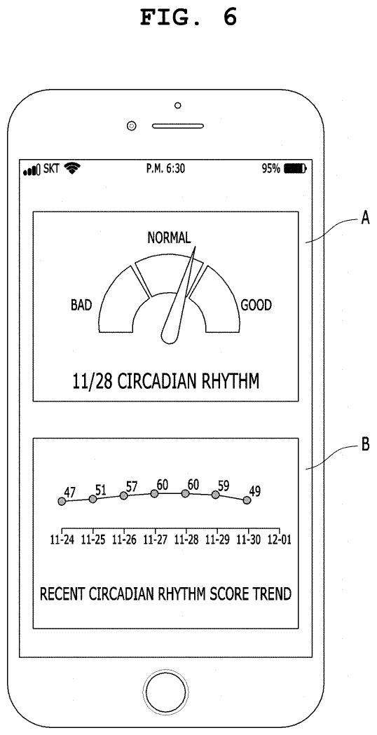

FIG. 6 is a view illustrating an interface expressed to provide evaluation information obtained by collecting user's health scores through a user terminal.

Referring to A of FIG. 6, the circadian rhythm management apparatus 100 may generate comprehensive health score evaluation information of the user 1, based on the calculated health scores for every category. For example, the circadian rhythm management apparatus 100 evaluates the comprehensive health scores based on the average information of the calculated health score for every category to display the circadian rhythm state of the user to be divided into “good”, “normal”, and “bad”, but is not limited thereto.

Further, referring to B (recent circadian rhythm score trend) of FIG. 6, the circadian rhythm management apparatus 100 may provide the comprehensive health score evaluation information of the user 1 predicted for a predetermined period as a time-sequential graph.

Further, according to the exemplary embodiment of the present disclosure, the circadian rhythm management apparatus 100 may provide a predetermined warning alarm to at least one of the user terminal 300 and the wearable device 200 when the health score is deteriorated to be lower than a predetermined level. For example, referring to A of FIG. 6, as a comprehensive evaluation result of the health score of the user 1, when the circadian rhythm state of the user 1 is deteriorated to a range corresponding to “bad”, a predetermined warning alarm may be displayed. For example, the warning alarm may be provided to the user 1 in various forms, such as a vibration, a warning voice/sound, a push message, SMS, or MMS.

To be more specific, when an average value for the calculated heart rate related score, light exposure related score, sleep related score, and activity related score is lower than a predetermined first threshold value, the circadian rhythm management apparatus 100 may operate to provide the warning alarm. As another example, when an average value for the heart rate related score, the light exposure related score, the sleep related score, and the activity related score calculated for a predetermined monitoring period is consistently lower than a predetermined second threshold value during the above-described monitoring period, the circadian rhythm management apparatus 100 may provide the warning alarm. Here, the second threshold value may be set to be higher than the first threshold value.

With regard to this, for example, when the health score is digitized (evaluated as a score in the range of 0 and 100) with 100 points as a perfect score, it is assumed that the first threshold value is 50 points and the second threshold value is 60 points. In this case, when the average of the health score calculated for every category is calculated to be equal to or lower than 50 points which is the first threshold value so that it is evaluated that the circadian rhythm of the user 1 is deeply deteriorated, the circadian rhythm management apparatus 100 immediately outputs a warning alarm. Further, even though the average of the health scores calculated for every category is not deteriorated to 50 points or less which are the first threshold value, when the level 60 points or less which is the second threshold value is consistently maintained for a predetermined monitoring period (for example, two days), the circadian rhythm management apparatus 100 may operate to output a warning alarm.

With regard to this, according to the implementation embodiment of the present disclosure, the warning alarm based on the first threshold value and the warning alarm based on the second threshold value may be output in different ways and have different contents.

Further, according to the exemplary embodiment of the present disclosure, the circadian rhythm management apparatus 100 may collect wearing hour information of the wearable device 200. For example, the wearing time information of the wearable device 200 may include daily wearing hours, weekly wearing hours, monthly wearing hours, hourly wearing hours, and the like of the wearable device 200. Further, the circadian rhythm management apparatus 100 may provide the feedback information based on the collected wearing hour information of the wearable device 200 of the user 1 through the user terminal 300.

For example, when the user's wearing hour information calculated daily, weekly, and monthly is below the predetermined threshold hour range, feedback information requesting to wear the wearable device 200 or increase the wearing hours may be output to the user terminal 300. With regard to this, the above-described threshold hour range which is a reference to output the feedback information provided with regard to the wearing hours of the wearable device 200 may be customized based on the gender, the age, the body type, the BMI information, the disease history, and the mood disorder of the user.

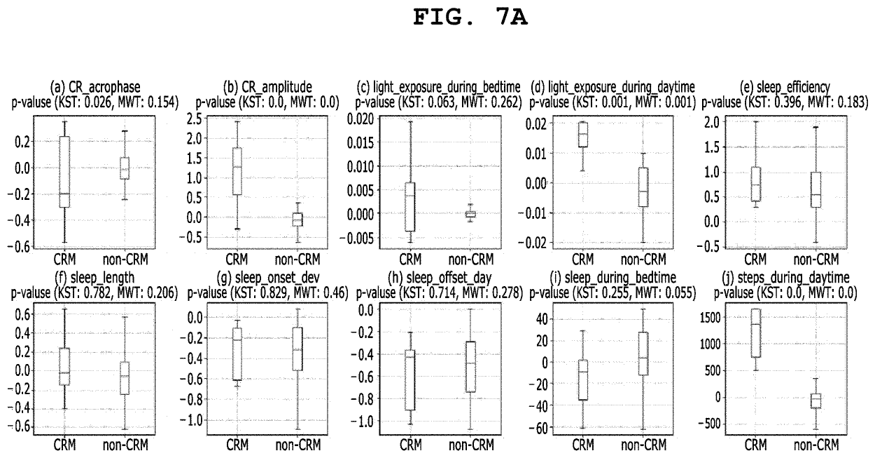

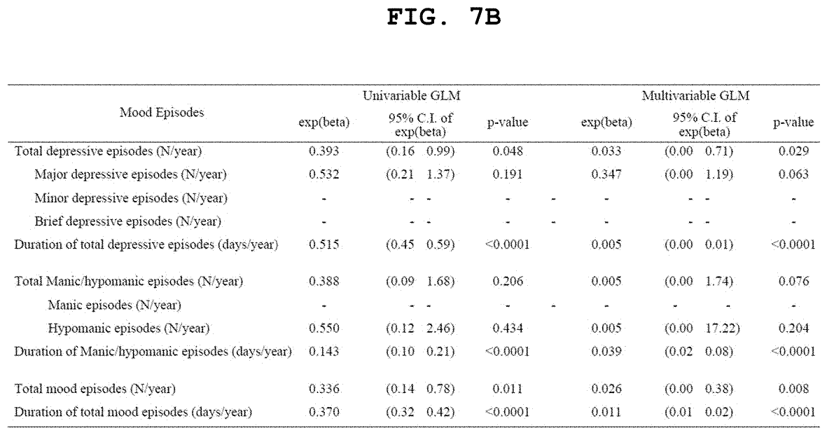

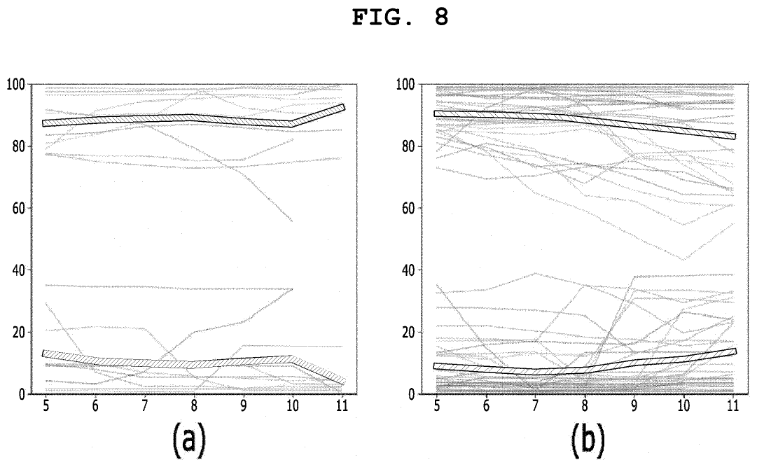

Hereinafter, an effect of the circadian rhythm management technique disclosed in the present disclosure will be described with the experiment result associated with the circadian rhythm management apparatus 100 according to the exemplary embodiment of the present disclosure with reference to FIGS. 7A, 7B, and 8.

The experimental embodiment associated with the circadian rhythm management apparatus 100 which will be described below is performed by comparing whether to improve the life pattern, generation of a mood episode, and a wearing rate of the wearable device 200 between a circadian rhythm for mood (CRM) group which is a group of patients who consistently receives the feedback information and the warning alarm through the circadian rhythm management apparatus 100 disclosed in the present disclosure while living an ordinary life and a non-CRM group which is a group of patient who lives the life without receiving the feedback information and the warning alarm through the circadian rhythm management apparatus 100, in a predetermined patient group with a mood disorder.

Further, in order to evaluate the generation of the mood episode in two groups (CRM group and non-CRM group), two groups are requested to perform self-mood evaluation (for example, create an eMoodChart) every day and the self-mood evaluation result performed by two groups are evaluated by a psychiatrist so that the occurrence tendency (a rate and a frequency) of the mood episode in two groups is analyzed.

FIGS. 7A and 7B are views illustrating a result of comparing feature information and mood episodes in a user group to which a feedback based circadian rhythm management of the present disclosure is applied and a user group to which the circadian rhythm management is not applied, as an experimental embodiment related to a circadian rhythm management apparatus based on a feedback function according to an exemplary embodiment of the present disclosure.

Specifically, FIG. 7A is a view illustrating comparison of the change distributions of the feature information separately acquired from the CRM group and the non-CRM groups. (a) to (j) of FIG. 7A are graphs obtained by comparing the change in the feature information corresponding to the acrophase (CR acrophase), the amplitude (CR amplitude), the light exposure amount during the bedtime (light exposure during bedtime), the light exposure amount during the daytime (light exposure during daytime), the quality of sleep (sleep efficiency), the sleep length (sleep length), the sleep onset deviation (sleep onset dev), sleep offset deviation (sleep offset dev), the step counts during the bedtime (steps during bedtime), and the step counts during the daytime (steps during daytime) between the CRM group and the non-CRM group). Specifically, as a median value shown in green is larger than 0, it may be interpreted that the value corresponding to the feature information is collected to show a tendency which increases in accordance with the elapse of time and in contrast, as the median value is smaller than 0, it may be interpreted that the value corresponding to the feature information is collected to show a tendency which decreases in accordance with the elapse of time. Further, KST and MWT may refer to Kolmogorov-Smirnov test and Mahn Whitney U test, respectively.

Referring to FIG. 7A, it may be confirmed that the change occurs to improve the feature information in the CRM group, as compared with the non-CRM group. Specifically, referring to (b), (d,), and (j) of FIG. 7A, the user in the CRM group improves a life style to a correct life pattern which gets sleep during the bedtime and mainly does activity during the daytime. Therefore, it may be confirmed that the heart rate function of the user in the CRM group is significantly improved.

Further, FIG. 7B compares a reoccurrence tendency of the mood episode in the CRM group and the non-CRM group. Referring to FIG. 7B, according to a univariable GLM analysis, it may be confirmed that as compared with the non-CRM group, total depressive episodes (N/year) of the CRM group is 60.7% smaller, the duration of total depressive episodes (days/year) is 48.5% shorter, a duration of manic/hypomanic episodes (days/year) is 85.7% shorter, the total mood episode occurrence frequency (N/year) is 66.4% less, and the duration of total mood episodes (days/year) is 63% shorter.

Further, referring to FIG. 7B, according to a multivariable GLM analysis, it may be confirmed that as compared with the non-CRM group, total depressive episodes (N/year) of the CRM group is 96.7% smaller, the duration of total depressive episodes (days/year) is 99.5% shorter, a duration of manic/hypomanic episodes (days/year) is 96.1% shorter, the total mood episode occurrence frequency (N/year) is 97.4% less, and the duration of total mood episodes (days/year) is 98.9% shorter.

FIG. 8 is a view illustrating a result of comparing a wearing rate of a wearable device in a user group to which a feedback based circadian rhythm management of the present disclosure is applied and a user group to which the circadian rhythm management is not applied, as an experimental embodiment related to a circadian rhythm management apparatus based on a feedback function according to an exemplary embodiment of the present disclosure.

Referring to FIG. 8, a wearing rate of the wearable device 200 are compared by calculating a moving average MA illustrated in an upper side and a moving standard deviation MSD illustrated in a lower side in the CRM group (FIG. 8A and the non-CRM group (FIG. 8B) for a total of 30 days with a window size of six days. A thin line indicates a trend line of patients in each group and a thick line indicates an average trend line of all the patients in each group. When the moving average MA shows a tendency which increases in accordance with the elapse of time and the trend line of the moving standard deviation MSD shows a tendency which decreases in accordance with the elapse of time, it may reflect that the wearable device 200 is consistently worn in the group.

With regard to this, referring to FIG. 8, the patients (users) belonging to the CRM group have a high wearing rate of the wearable device 200 within the corresponding period and consistently maintains the worn state during this period. In contrast, it may be confirmed that the wearing rate of the wearable device 200 of the patients (users) belonging to the non CRM group decreases in accordance with the elapse of time during the corresponding period and this tendency is accelerated.

From the above-described experimental embodiment associated with the circadian rhythm management apparatus 100 according to the exemplary embodiment of the present disclosure, it may be confirmed that when the mood prediction result of the user 1, the health score analysis result, the feedback information including a behavioral pattern to improve the health score, and the warning alarm are consistently monitored and provided based on a circadian rhythm by the circadian rhythm management apparatus 100, it is possible to induce the user 1 to live a regular daily life and thus assist the user to improve the circadian rhythm of the user 1. Specifically, an actual behavior change of a patient with a mood disorder is triggered and the wearing of the wearable device 200 is consistently maintained to effectively prevent the recurrence of the mood episode and achieve a positive effect on the rapid recovery even after the recurrence of the mood disorder.

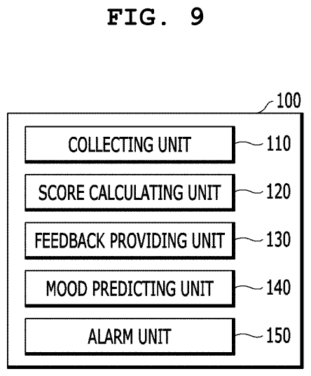

FIG. 9 is a schematic diagram of a circadian rhythm management apparatus based on a feedback function according to an exemplary embodiment of the present disclosure.

Referring to FIG. 9, the circadian rhythm management apparatus 100 may include a collecting unit 110, a score calculating unit 120, a feedback providing unit 130, a mood predicting unit 140, and an alarm unit 150.

The collecting unit 110 may collect the feature information including heart rate information, light exposure information, sleep information, and step information for the user 1. Further, the collecting unit 110 may collect wearing hour information of the wearable device 200.

The score calculating unit 120 may calculate a health score for the user 1 based on the collected feature information.

Specifically, the score calculating unit 120 may calculate a heart rate related score of the user 1 based on at least one of an acrophase, an amplitude, a vibration mesor and a coefficient of determination included in the heart rate information. Further, the score calculating unit 120 may calculate the light exposure related score of the user 1 based on at least one of the light exposure amount during the daytime and the light exposure amount during the bedtime included in the light exposure information. Further, the score calculating unit 120 may calculate the sleep related score of the user 1 based on at least one of the sleep length, a quality of sleep, a sleep onset deviation, and a sleep offset deviation included in the sleep information. Further, the score calculating unit 120 may calculate the activity related score of the user 1 based on at least one of cumulative step counts during the daytime and cumulative step counts during the bedtime included in the step information.

The feedback providing unit 130 may provide feedback information including a behavioral pattern to improve the health score of the user 1 based on the calculated health score to at least one of the user terminal 300 and the wearable device 200. Further, the feedback providing unit 130 may provide the feedback information based on the collected wearing hour information of the wearable device 200 through the user terminal 300.

The mood predicting unit 140 may generate mood change prediction information of the user 1 corresponding to feature information collected for the user 1, based on a prediction model which is trained in advance to output mood change prediction information corresponding to the input feature information. Further, the mood predicting unit 140 may provide the generated mood change prediction information to at least one of the user terminal 300 or the wearable device 200.

The alarm unit 150 may provide a predetermined warning alarm to at least one of the user terminal 300 and the wearable device 200 when the calculated health score of the user 1 is deteriorated to be lower than a predetermined level.

Hereinafter, an operation flow of the present disclosure will be described in brief based on the above detailed description.

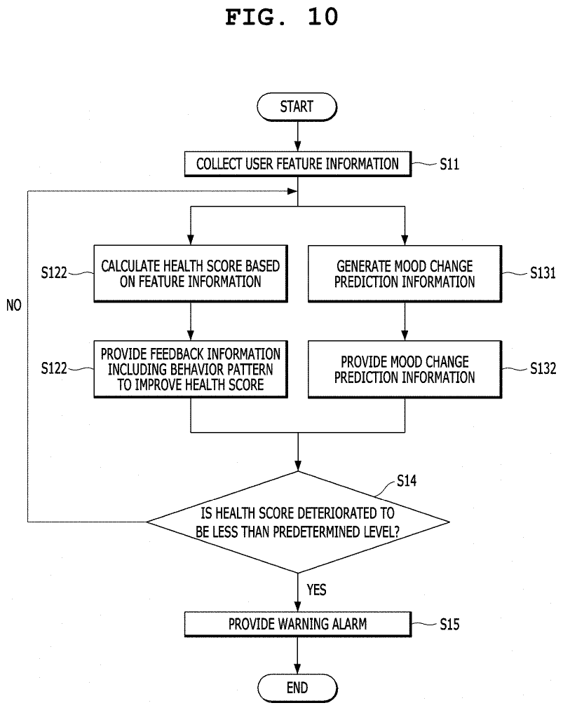

FIG. 10 is a flowchart of an operation of a circadian rhythm management method based on a feedback function according to an exemplary embodiment of the present disclosure.

The circadian rhythm management method based on a feedback function illustrated in FIG. 10 may be performed by the circadian rhythm management apparatus 100 which has described above. Therefore, even though some contents are omitted, the contents which have been described for the circadian rhythm management apparatus 100 may be applied to the description of the circadian rhythm management method based on a feedback function in the same manner.

Referring to FIG. 10. In step S11, the collecting unit 110 may collect the feature information including heart rate information, light exposure information, sleep information, and step information for the user 1 from at least one of the user terminal 300 and the wearable device 200.

Further, in step S11, the collecting unit 100 may collect the exposure information and the step information separately for the plurality of time slots which has been divided in advance.

Next, in step S121, the score calculating unit 120 may calculate a health score for the user 1 based on the collected feature information.

Specifically, in step S121, the score calculating unit 120 may calculate a heart rate related score of the user 1 based on at least one of an acrophase, an amplitude, a vibration mesor and a coefficient of determination included in the heart rate information. Further, in step S121, the score calculating unit 120 may calculate the light exposure related score of the user 1 based on at least one of the light exposure amount during the daytime and the light exposure amount during the bedtime included in the light exposure information. Further, in step S121, the score calculating unit 120 may calculate the sleep related score of the user 1 based on at least one of the sleep length, the quality of sleep, a sleep onset deviation, and a sleep offset deviation included in the sleep information. Further, in step S121, the score calculating unit 120 may calculate the activity related score of the user 1 based on at least one of cumulative step counts during the daytime and cumulative step counts during the bedtime included in the step information.

Next, in step S122, the feedback providing unit 130 may provide feedback information including a behavioral pattern to improve the health score based on the calculated health score to at least one of the user terminal 300 and the wearable device 200.

Further, in step S131, the mood predicting unit 140 may generate mood change prediction information of the user 1 corresponding to feature information collected for the user 1, based on a prediction model which is trained in advance to output mood change prediction information corresponding to the collected feature information.

Next, in step S132, the mood predicting unit 140 may provide the generated mood change prediction information to at least one of the user terminal 300 or the wearable device 200. Specifically, in step S132, the mood predicting unit 140 may display a virtual avatar whose outer appearance is determined in response to the generated mood change prediction information on at least one of the user terminal 300 and the wearable device 200.

Next, in step S14, the alarm unit 150 may determine whether the calculated health score of the user 1 is deteriorated to be lower than a predetermined level.

Specifically, in step S14, the alarm unit 150 may determine whether an average value for the heart rate related score, the light exposure related score, the sleep related score, and the activity related score is lower than a predetermined first threshold value.

Further, in step S14, the alarm unit 150 may determine whether an average value for the health scores during the predetermined monitoring period is consistently lower than a predetermined second threshold value which is set to be higher than the first threshold value.

Next, in step S15, when it is determined that the health score of the user is deteriorated to be lower than a predetermined level as compared with at least one of the first threshold value and the second threshold value in step S14, the alarm unit 150 may provide a predetermined warning alarm to at least one of the user terminal 300 and the wearable device 200.

In the above-description, steps S11 to S15 may be further divided into additional steps or combined as smaller steps depending on an implementation embodiment of the present disclosure. Further, some steps may be omitted if necessary and the order of steps may be changed.

The method for managing a circadian rhythm based on a feedback function according to the exemplary embodiment of the present invention may be implemented as a program command which may be executed by various computers to be recorded in a computer readable medium. The computer readable medium may include solely a program instruction, a data file, and a data structure or a combination thereof. The program instruction recorded in the medium may be specifically designed or constructed for the present disclosure or known to those skilled in the art of a computer software to be used. Examples of the computer readable recording medium include a magnetic media such as a hard disk, a floppy disk, or a magnetic tape, an optical media such as a CD-ROM or a DVD, a magneto-optical media such as a floptical disk, and a hardware device which is specifically configured to store and execute the program instruction, such as a ROM, a RAM, and a flash memory. Examples of the program instruction include not only a machine language code which is created by a compiler but also a high level language code which may be executed by a computer using an interpreter. The above-described hardware device may operate as one or more software modules in order to perform the operation of the present disclosure and vice versa.

Further, the above-described method for managing a circadian rhythm based on a feedback function may also be implemented as a computer program or an application executed by a computer which is stored in a recording medium.

The above description of the present disclosure is illustrative only and it is understood by those skilled in the art that the present disclosure may be easily modified to another specific type without changing the technical spirit of an essential feature of the present disclosure. Thus, it is to be appreciated that the embodiments described above are intended to be illustrative in every sense, and not restrictive. For example, each component which is described as a singular form may be divided to be implemented and similarly, components which are described as a divided form may be combined to be implemented.

The scope of the present disclosure is represented by the claims to be described below rather than the detailed description, and it is to be interpreted that the meaning and scope of the claims and all the changes or modified forms derived from the equivalents thereof come within the scope of the present disclosure.

What is claimed is:

1. A circadian rhythm management method based on a feedback function, comprising:
collecting feature information including a heart rate information, light exposure information, sleep information, and step information for a user;
calculating a health score for the user based on the feature information; and
providing feedback information including a behavior pattern to improve the health score based on the calculated health score through a user terminal of the user.

2. The circadian rhythm management method according to claim 1, wherein in the collecting of feature information, the light exposure information and the step information are separately collected for each of a plurality of time slots which is divided in advance.

3. The circadian rhythm management method according to claim 2, further comprising:
generating mood change prediction information of the user corresponding to the feature information based on a prediction model which is trained in advance to output the mood change prediction information corresponding to the input feature information; and
providing the mood change prediction information through the user terminal.

4. The circadian rhythm management method according to claim 3, wherein in the providing of the mood change prediction information, a virtual avatar whose outer appearance is determined in response to the mood change prediction information is displayed on the user terminal.

5. The circadian rhythm management method according to claim 2, wherein the heart rate information includes an acrophase, an amplitude, a mesor of the vibration, and a coefficient of determination in a pulse fitting curve derived based on the pulse rate per unit time of the user,
the light exposure information includes a light exposure amount during the daytime and a light exposure amount during the bedtime;
the sleep information includes a sleep length, a sleep quality, a sleep onset deviation, and a sleep offset deviation, and
the step information includes cumulative step counts during the daytime and cumulative step counts during the bedtime.

6. The circadian rhythm management method according to claim 5, wherein the calculating of the health score includes:
calculating a heart rate related score of the user based on at least one of the acrophase, the amplitude, the mesor of the vibration, and the coefficient of determination;
calculating a light exposure related score based on at least one of the light exposure amount during the daytime and the light exposure amount during the bedtime;
calculating a sleep related score based on at least one of the sleep length, the sleep quality, the sleep onset deviation, and the sleep offset deviation; and
calculating an activity related score based on at least one of the cumulative step counts during the daytime and the cumulative step counts during the bedtime.

7. The circadian rhythm management method according to claim 6, further comprising:
providing a predetermined warning alarm to the user when the health score is deteriorated to be lower than a predetermined level.

8. The circadian rhythm management method according to claim 7, wherein in the providing of the warning alarm, when an average value of the heart rate related score, the light exposure related score, the sleep related score, and the activity related score is lower than a predetermined first threshold value, the warning alarm is provided.

9. The circadian rhythm management method according to claim 8, wherein in the providing of the warning alarm, when the average value for a predetermined monitoring period is maintained to be lower than a predetermined second threshold value, the warning alarm is provided and the second threshold value is set to be higher than the first threshold value.

10. The circadian rhythm management method according to claim 1, wherein in the collecting of the feature information, the feature information is collected from at least one of the user terminal and a wearable device which is worn on a body of the user.

11. The circadian rhythm management method according to claim 10, further comprising:
collecting wearing hour information of the wearable device; and
providing feedback information based on the wearing hour information to the user terminal.

12. A circadian rhythm management apparatus based on a feedback function, comprising:
a collecting unit which collects feature information including a heart rate information, light exposure information, sleep information, and step information for a user;
a score calculating unit which calculates a health score for the user based on the feature information; and
a feedback providing unit which provides feedback information including a behavior pattern to improve the health score based on the calculated health score through a user terminal of the user.

13. The circadian rhythm management apparatus according to claim 12, further comprising:
a mood predicting unit which generates a mood change prediction information of the user corresponding to the feature information based on a prediction model trained in advance to output the mood change prediction information corresponding to the input feature information and provides the mood change prediction information by means of the user terminal.

14. The circadian rhythm management apparatus according to claim 12, wherein the heart rate information includes an acrophase, an amplitude, a mesor of the vibration, and a coefficient of determination in a pulse fitting curve derived based on the pulse rate per unit time of the user,
the light exposure information includes a light exposure amount during the daytime and a light exposure amount during the bedtime;
the sleep information includes a sleep length, a sleep quality, a sleep onset deviation, and a sleep offset deviation, and
the step information includes cumulative step counts during the daytime and cumulative step counts during the bedtime;
the score calculating unit calculates a heart rate related score of the user based on at least one of the acrophase, the amplitude, the mesor of the vibration, and the coefficient of determination, calculates a light exposure related score based on at least one of the light exposure amount during the daytime and the light exposure amount during the bedtime; calculates a sleep related score based on at least one of the sleep length, the sleep quality, the sleep onset deviation, and the sleep offset deviation, and calculates an activity related score based on at least one of the cumulative step counts during the daytime and the cumulative step counts during the bedtime.

15. The circadian rhythm management apparatus according to claim 14, further comprising:
an alarm unit which provides a predetermined warning alarm to the user terminal when the health score is deteriorated to be lower than a predetermined level.
